(19) United States
(12) Patent Application Publication
(10) Pub. No.: US 2014/0349329 A1
(43) Pub. Date: Nov. 27, 2014

(54) DENSITY ANALYSIS OF ORGANISMS BY MAGNETIC LEVITATION

(71) Applicant: President and Fellows of Harvard College, Cambridge, MA (US)

(72) Inventors: George M. WHITESIDES, Cambridge, MA (US); Anna LAROMAINE SAGUÉ, Cambridge, MA (US); Ratmir DERDA, Cambridge, MA (US); Charles R. MACE, Auburn, NY (US); Katherine A. MIRICA, Waltham, MA (US); Alfonso Reina CECCO, Cleveland Heights, OH (US); Suzanne HULME, Cambridge, MA (US)

(73) Assignee: President and Fellows of Harvard College, Cambridge, MA (US)

(21) Appl. No.: 14/223,963

(22) Filed: Mar. 24, 2014

Related U.S. Application Data
(63) Continuation of application No. PCT/US2012/056655, filed on Sep. 21, 2012.
(60) Provisional application No. 61/538,442, filed on Sep. 23, 2011.

(51) Int. Cl. G01N 33/50

(52) U.S. Cl. CPC G01N 33/5085; G01N 33/5088
USPC 435/29; 435/288.7

(57) ABSTRACT

A device and methods for detecting the effect of compounds on an organism are provided. Furthermore, the device and methods disclosed herein allow for the fractionation of complex samples and the isolation of one or more organisms for the samples. The device and methods also allow for the study of development of the organism.

### CROSS-REFERENCE TO RELATED APPLICATIONS

This application is a continuation of International Application No. PCT/US2012/056655, which was filed on Sep. 21, 2012, which claims priority to U.S. Provisional Application Ser. No. 61/538,442, which was filed on Sep. 23, 2011. These applications are hereby incorporated by reference in their entirety.

This disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

All patent applications, published patent applications, issued and granted patents, texts, and literature references cited in this specification are hereby incorporated herein by reference in their entirety to more fully describe the state of the art to which the present invention pertains.

### FIELD OF THE INVENTION

The invention is generally directed to methods of analyzing and separating complex samples. Specifically, the invention is directed to methods of analyzing organisms in biological samples.

### BACKGROUND OF THE INVENTION

The study of microscopic organisms requires the ability to separate such organisms from complex samples. Separation techniques must allow researchers to differentiate between organisms of interest and the rest of the sample. Furthermore, certain studies require that the separation of the organisms not damage or kill the organisms.

One of the characteristics of magnetic levitation is that the levitation height of an object is directly related to its density, and thus there is only one position in the magnetic field in which an object is stably levitated. When a levitating object in the magnetic field is moved away from a position of equilibrium, a restoration force on the object returns it to equilibrium position. Therefore, a mixture of substances—each with a unique density—will levitate at different levitation heights in the same magnetic field, and can thus be separated.

Past techniques have not allowed for simple analysis of density in real-time. In addition, previous analytical techniques have been not amenable to the analysis of changes in density of an object, such as a living organism. Thus, these techniques do not allow researchers to study the growth rate of organisms, their development (i.e., developmental characteristics that are associated with density), or other characteristics associated with the life of an organism of interest.

Therefore, there is a need for methods to separate organisms from other components in a complex sample without damaging or killing the organism. Furthermore, there remains a need for methods that allow for the assaying of the effects of compounds of interest on organisms.

### SUMMARY OF THE INVENTION

According to aspects of the present disclosure, methods and devices are disclosed that allow for the separation and/or isolation of organisms from other components in a sample. In addition, the disclosed devices and techniques allow for the analysis of changes in density of an object in real time. Furthermore, the disclosed devices and techniques allow for monitoring of density changes of an object, such as an organism. The methods and devices utilize magnetic levitation to separate and/or isolate the organisms by their density. In addition, methods are disclosed herein that allow for the analysis of the toxicity of compounds and/or the effects of compounds on an organism. Furthermore, the methods and devices disclosed herein are useful for the analysis of the early development of a multicellular organism.

Aspects disclosed herein include methods for detecting an effect of a compound of interest on a biological system. The methods comprise contacting a test sample comprising an organism with the compound of interest (e.g., toxins, drugs, or particles) in a paramagnetic solution and applying a magnetic field to the test sample. The methods also entail determining the density of the living organism in the test sample. In these aspects, the organism occupies a position in the magnetic field that is an indication of its density. In certain embodiments, the methods comprise comparing the density or location of the organism in the test sample to a reference density or to the location of an untreated reference organism and detecting the effect of the compound of interest on a biological condition based on a change in density of the organism.

In other embodiments, the location of the living organism in the test sample is determined at different time points. In further embodiments, the change in density in the organism is an indication of altered fat content when the organism is in the presence of the compound of interest. In still further embodiments, the change in density in the organism is indicative of uptake and accumulation of the compound of interest by the organism. In other embodiments, a change in density in the organism is an indication of altered water content when the organism is in the presence of the compound of interest.

In some embodiments, the methods further comprise providing a plurality of test samples comprising the organism and introducing a different compound of interest into each of the plurality of test samples. The methods also further comprise identifying those test samples containing organism contacted with the different compound of interest that demonstrate a change in density or location relative to the reference density or location of a reference organism that is not contacted with the different compound of interest. In these embodiments, the change in density or location is indicative of a biological effect on the organism by the compound of interest.

In particular embodiments, the organism is an embryo of a multicellular organism. In more particular embodiments, detecting the effect of the compound of interest involves noting a change in embryonic development. In certain embodiments, detecting the effect of the compound of interest involves noting changes in the movement of an organism. In particular embodiments, detecting the effect of the compound of interest involves noting changes in the swimming rate of the organism.

Aspects of disclosed herein include methods for determining the toxicity of a compound on a biological system. The methods comprise contacting a plurality of test samples comprising an organism to a compound of interest at increasing concentrations and applying a magnetic field to the test samples. The methods also entail determining the density of the organism in the each of the plurality of test samples, wherein the organism occupies a position in the magnetic field that is an indication of its density and identifying the density in the test sample with a level of altered fat content of the organism, wherein a preselected level of fat content is associated with toxicity. The methods further include determining a concentration of the compound of interest that provides a density change in the organism associated with toxicity.

Still more aspects include methods of evaluating an embryo. The methods comprise exposing a paramagnetic solution comprising an embryo to a magnetic field. The embryo occupies a position in the magnetic field that is an indication of its density. In addition, the methods comprise monitoring the position of the embryo with time and detecting a change in location over time, the change in location being associated with gestational development of the embryo.

In certain embodiments, the change in density or position identifies a change in gestational growth rate.

Further aspects disclosed herein involve methods of sorting a population of organisms. The methods comprise exposing a paramagnetic solution comprising a population of organisms to a magnetic field. The individual members of the population occupy positions in the magnetic field that correspond to their densities. The methods also include sorting the population by density, based on its position in the magnetic field.

In certain embodiments, the methods further comprise isolating the population from the paramagnetic solution.

Aspects disclosed herein also include methods of analyzing a sample for the presence of an organism. The methods comprise exposing a test sample in a paramagnetic solution to a magnetic field and determining positions in the magnetic field of one or more constituent components of the test sample, wherein the positions are characteristic of their densities. The methods also comprise detecting the presence or absence of a component at a predetermined position in the magnetic field that is associated with the presence or absence of the organism in the test sample.

In certain embodiments, the sample is a biological sample. In other embodiments, the biological sample is selected from the group consisting of bodily fluids and body tissues. In still other embodiments, the organism has been preselected based on a characteristic of the organism. The characteristic includes, but is not limited to, fatty acid metabolism or other metabolic factors, biological factors such as infectivity or parasitic characteristics, and developmental factors, such as gestation time. In particular embodiments, the preselected organism is a parasite and the presence of the organism in the sample is indicative of parasitic infection.

Aspects provided herein also include methods of analyzing an organism of interest. The methods comprise providing a paramagnetic solution of a composition and osmolality compatible with an organism of interest. The methods further entail introducing the organism of interest into the paramagnetic solution and applying a magnetic field to the paramagnetic solution. In certain embodiments, the methods entail detecting the density of the organism of interest by determining the position of the organism of interest in the magnetic field.

In other embodiments, the paramagnetic solution comprises a chelated metal salt. In still other embodiments, the chelated paramagnetic salt comprising manganese or gadolinium. In further embodiments, the paramagnetic solution further comprises a paralyzing agent. In still other embodiments, the paramagnetic solution is at a temperature lower than the optimal temperature of the organism. Such optimal temperatures are lower than the temperature required for optimal cellular functions. In certain embodiments, the temperature of the paramagnetic solution is 4° C. In still further embodiments, the organism is selected from the group consisting of prokaryotic cells, eukaryotic cells, parasitic worms, ova, embryos and spermatozoa. In other embodiments, the organism is a plant tissue, a seed, a seedling, a tumor, a cancer mass, a group of cells, a spore, a pollen granule, a worm, or a multicellular parasite.

Further aspects disclosed herein provide devices for determining the effect of a compound on a biological system. Such devices are also used to measure density of organisms. The devices comprise a pair of permanent magnets positioned to provide a magnetic field of a predetermined field gradient. The devices also comprise a sample holder located within the magnetic field for receiving a sample comprising a living organism and a scale affixed to the magnet pair for use in determining the relative and/or absolute positions of living organisms viewable in a sample. In certain embodiments, the device is configured to receive a sample comprising a suspension of living organisms housed in a microfluidic chip.

### DESCRIPTION OF THE FIGURES

The following figures are presented for the purpose of illustration only, and are not intended to be limiting:

FIG. 1 is a schematic representation (A) of the magnetic field, (B) the distribution of magnetic forces, and (C) a graph of the calculated magnitude of magnetic field along the axis of the magnets used for separation.

FIG. 2 is a schematic illustration of a device for determining the location of a diamagnetic particle in paramagnetic solution exposed to a magnetic force.

FIG. 3 shows experiments determining the effects on the density of C. elegans after administration of aspirin.

FIG. 4 shows the changes in density associated with different time points in the development of Danio rerio (i.e., zebrafish).

FIG. 5a shows the structure of a microfluidic device used in magnetic levitation assays.

FIG. 5b shows how C. elegans pass through the microfluidic device.

FIG. 6a shows two microfluidic chambers. The left chamber is loaded with C. elegans and paramagnetic solution. The chambers were placed between two magnets.

FIG. 6b shows a chamber loaded with C. elegans and paramagnetic solution after 15 min (left) and 60 min (right) of being placed between the magnets. The C. elegans start levitating and adopting an equilibrium position.

FIG. 7 shows a simplified schematic of a microfluidic device.

### DETAILED DESCRIPTION

### 1. General

According to aspects of the present disclosure, devices and methods for separating or isolating an organism from a solution are described. As used herein, the term “organism” means a form of life—unicellular or multicellular—that exhibits one or more attributes of life (i.e., metabolism, reproduction, etc.). Examples of organisms include prokaryotic organisms, such as bacteria, single cell eukaryotic organisms, such as protists (e.g., Plasmodium, algae, amoeba), cells from multicellular organisms, such as ova, spermatozoa, and cells from tissues, as well as fungi and other small multicellular organisms such as C. elegans. The techniques disclosed herein comprise exposing a paramagnetic solution comprising an organism (e.g., an embryo) to a magnetic field. The diamagnetic characteristics of the organism force the organism to occupy a position in the magnetic field. As is described below, the position that the organism occupies in the solution, the ‘levitation height’, correlates with its density. Thus, the organism is levitated into a particular position within the paramagnetic solution and is separated from other cells or materials in the sample that are of a different density.

As is apparent to one of ordinary skill in the art, this technique allows for isolation of the cells that have been separated according to the above method. This can be accomplished by removing the desired cells via means that are known in the art. Such means include aspiration of the organism of interest using a needle attached to an aspirator or removal of unwanted layers of material until the “band” containing the organism has been reached. Furthermore, needle aspiration can be performed by inserting a needle attached to a syringe through the side of the container used during the experiment. The insertion of the needle should be accomplished in such a way as to avoid disturbing the paramagnetic solution when removing the organisms. Such needles should be diamagnetic. In some embodiments, the organisms are pre-stained with a non-toxic fluorescent label prior to separation in the solution to enable visualization of the band. Membrane-specific lipid and protein fluorescent labels and probes can be obtained commercially, for example, from Sigma-Aldrich Corp. (St. Louis, Mo.).

When determining the density of labeled cells, control cells can be used. The control cells are cells that are not treated with a compound and are not labeled with the probe or label that was used for visualization of the cells.

Additionally, continuous flow cell separation techniques can be employed to separate and isolate the cells. Techniques utilizing density differences are known in the art (see, e.g., Ito et al. (2001) J Clin Apher. 16(4): 186-91). In the disclosed methods, a microfluidic device can be utilized. The microfluidic device includes components on the order of micrometers to centimeters that are designed to handle fluid flow. In some embodiments, a pump may be used to maintain a fluid flow. In other embodiments, the microfluidic device can work without the need of electrical power (with gravity as the only pumping force of the system) thus providing a means for automating separation and collection processes at very high volumes (thousands of liters) while keeping the cost of the process extremely low, since the paramagnetic solution can be reused. This technique could be useful in recycling processes where different organisms could be continuously separated as a function of their density and in processes that want to avoid the need of expensive reagents like antibodies.

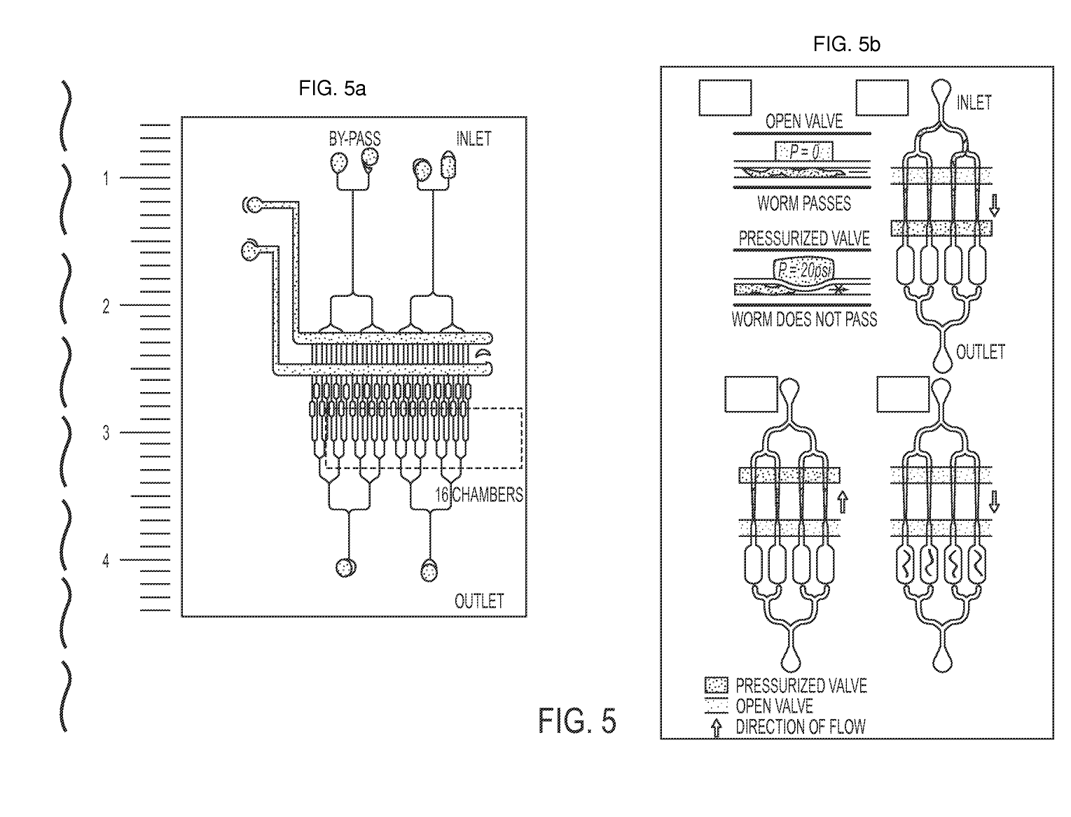

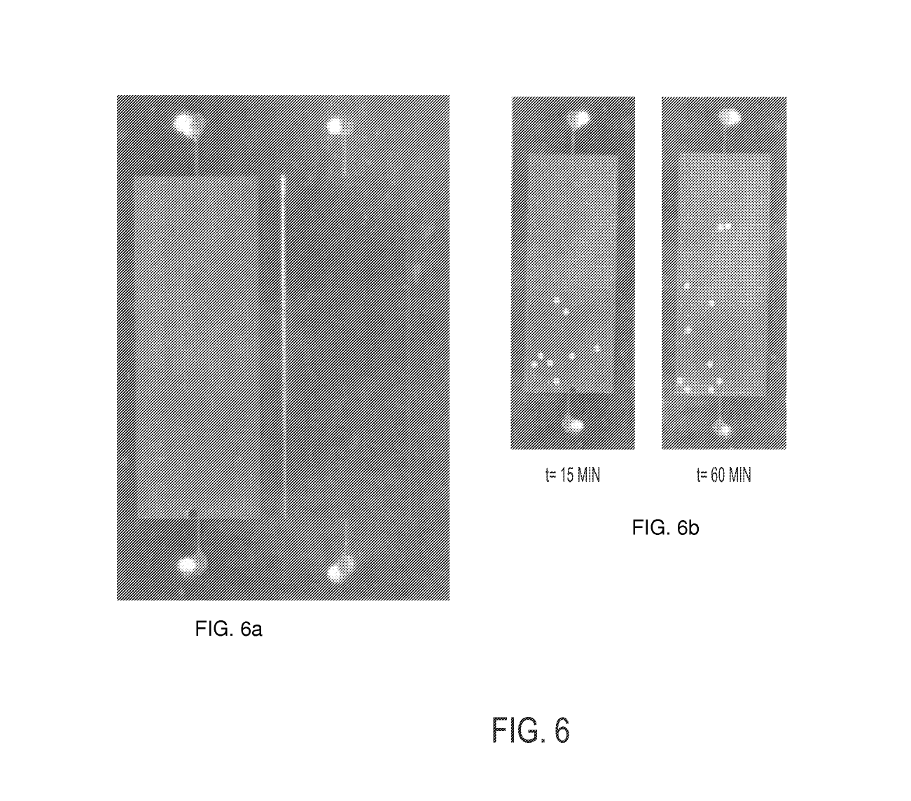

The microfluidic device takes advantage of laminar flow, that is, fluids flow in streams without turbulence that would disrupt separations. Microfluidic devices can allow for analysis of multiple organisms at once (FIGS. 5a and 5b). A microfluidic device for use according to one or more embodiments does not include magnetic components (except for the magnets used to generate a magnetic field), provides for the continuous flow and separation of materials in dimensions ranging from a few micrometers to a few centimeters, and is transparent or accessible to wavelengths used for detection (e.g., visible, ultraviolet, infrared). Microfluidic systems also use small volumes of sample and solution. In one of the embodiments, the microfluidic device is positioned between two magnets and includes at least one channel that traverses the magnetic field generated by the magnets. In certain embodiments, the microfluidic system is made of a polymer that is inert to the fluid flowing within the device. Such devices are disclosed in PCT Appl. Ser. No. US08/68797, the contents of which are incorporated by reference.

The organisms to be separated flow into the channel that is disposed within the magnetic field. The organisms are pumped into the chamber in a direction that is substantially orthogonal to the gradient of magnetic field. As the organisms move into the channel (perpendicularly to the gradient of magnetic field), they also migrate in the direction of the magnetic field gradient to an equilibrium position of levitation in the chamber that is a function of the applied magnetic field, the magnetic susceptibility of the solution, and the organism density. The organisms continue to flow through the chamber and pass at the opposite end into one of a plurality of outlet conduits that are positioned along the edge of the chamber in the direction perpendicular to that of the magnetic field gradient. The conduits collect the organisms after they have been separated in the channel and into a collection vial. In this way, solutions enriched with an organism of a specific density are obtained. The device can be manually or automatically operated. In some embodiments, it can be computer-controlled. The device can be scaled to accommodate samples in a range of sizes and volumes. By changing the size of the separating chamber, the paramagnetic strength of the dynamic fluid and the size and strength of the magnetic field, samples of varying sizes, organism sizes and amounts may be separated.

In some embodiments, the separation and/or isolation techniques further involve the use of density standard references that are used to determine the position that a particular density will assume in the magnetic field. Such references can be added to the sample to be separated or can be in a separate sample so long as the sample is subjected to a similar magnetic field and a solution of similar paramagnetic strength. The references are then used to determine the density of the organism or to identify the position that the organism should assume.

Reference standards can also be particles of known or identified densities. Any bead or particle of regular or irregular shape can be used, provided that it is diamagnetic and of a density that permits its displacement in a magnetic field. Suitable materials are not soluble in the solvent, do not react with the solvent, and do not swell to any considerable extent in the solvent, allowing for accurate density determinations. Exemplary polymer particles include particles made up of polystyrene, polypropylene, polyethylene, a Tentagel resin, an Argopore resin, polyethylene glycol (and copolymers of), polyacrylamide, poly(methyl methacrylate), and others.

The methods and devices disclosed herein can also be used to monitor the development of an organism. For instance, a fertilized egg of a multicellular organism can be isolated and its development monitored. At various time points during the development of the egg into a multicellular embryo, the embryo is subjected to a magnetic field and the position of the embryo is identified. Over time, the change in density of the embryo is monitored. Such changes in density are associated with differences in cell number, lipid content, and other factors. In other words, by detecting a change in density of the embryo (i.e., the location of the embryo in the magnetic field) over time, one monitors the gestational development of the embryo.

The methods and devices disclosed herein can also be used to detect the effects of compounds (e.g., pain-relieving drugs, therapeutics, antibiotics, pesticides, pollutants) on an organism. Such effects include, but are not limited to, developmental effects, such as delays in development, changes in growth rate, growth arrest, and death. The methods comprise contacting a test sample that has one or more organisms with the compound of interest. The organism can be incubated with the compound for any period of time that is required for the compound to have an effect. In addition, the methods allow for time points to be taken so that the effect of the compound on an organism can be determined over time. The organism can be contacted with the compound in a medium that is optimal for growth and development. Alternatively, the organism can be contacted with the compound in the paramagnetic solution.

These methods can also be used to determine the toxicity of compounds on a biological system (i.e., an organism). In certain embodiments, the methods employ a series or plurality of test samples, each of which comprises an organism that is contacted with a particular concentration of a compound of interest. This methodology involves exposing or applying the test samples to a magnetic field. In these embodiments, density changes correlate to alterations in nucleic acid content, lipid metabolism, or lipid content. In certain embodiments, the change in lipid metabolism is predetermined and selected as establishing a toxicity of the compound of interest. In other embodiments, a concentration of the compound of interest is identified that provides the greatest toxic effect to the organism. In more embodiments, a concentration is identified that has the least toxicity on the organism.

After the organism has been contacted with the compound, a magnetic field is applied to the sample containing the organism. The magnetic field can be applied contemporaneously with the contacting of the organism to the compound. The density of the organism in the test sample is determined by identifying the position in the magnetic field that the organism occupies. As described above, this can be accomplished by using reference standards, which include control samples where the organism was not treated with a compound or was subjected to a vehicle. The position corresponds to the organism's density and is further an indication that the compound had an effect on a biological condition (e.g., developmental, growth, or death).

The methods described herein can be utilized with any sample container that is composed of non-magnetic material such as polyethylene. In particular embodiments, the samples can be separated in test tubes, cuvettes, or multiwell plates. In certain embodiments, the wells of the multiwell plate should be of sufficient height or length to allow for separation or identification of organisms.

In addition, the methods provided herein can be used with non-toxic paramagnetic solutions. Such solutions can comprise paramagnetic salt chelates that are FDA-approved for use in subjects. Exemplary paramagnetic salts include manganese salts and gadolinium salts. In particular embodiments, the salts are chelated using an agent such as EDTA. It has been observed that chelated manganese salts are less toxic than chelated gadolinium salts, which must be used at low concentrations (<300 mM) to reduce toxicity, thereby placing very specific boundary conditions to the assay. Furthermore, the solutions can be isotonic to further decrease the effects of the solution on the organism. Isotonicity is determined with reference to the organism and such solutions can have a wide range of tonicities. Exemplary isotonic solutions have tonicities of 270-330 mOsm/kg. In certain embodiments, the solution has a tonicity of 300 mOsm/kg.

In additional embodiments, the solution comprises a compound to paralyze the organism to prevent movement. Exemplary paralyzing compounds include, but are not limited to, ivermectin, levamisole, muscimol, and sodium azide. In addition, it is useful to lower the temperature of the paramagnetic solution to a temperature that is less than optimal for the organism. In some instances, the temperature of the paramagnetic solution is decreased to 4° C. In other embodiments, the temperature of the paramagnetic solution is decreased to 0° C. or lower.

Disclosed herein are also methods of analyzing a sample for the presence of an organism. In certain embodiments, the sample is isolated from an environmental source such as water, soil, or surfaces. In other embodiments, the sample is isolated from a biological system, that is, from bodily fluids, tissues, or excretions (e.g., urine, fecal). The sample is prepared such that any large solid materials are removed using methods known in the art and suitable for the particular sample. The samples are then exposed to a magnetic field and the positions of one or more constituent components of the test sample can be identified at predetermined positions. The positions are predetermined by reference to a known density of the organism. The known density is determined prior to or during the experiments performed on the test samples. In certain embodiments, the known density is determined with reference to commercially available cells (American Type Culture Collection, Manassas, Va.). In other embodiments, the cells are isolated from a source and identified using other biological markers (e.g., proteins, genetic markers, etc.) using techniques known to those of ordinary skill in the art (see, e.g., Sambrook et al. Molecular Cloning: A Laboratory Manual (Third Edition). Cold Spring Harbor Laboratory Press.)

Additionally, the constituent components can be organisms such as bacterial organisms, such as E. coli in water tests, or parasites, such as fungi. The organisms can also be cancer cells isolated from a tissue of a subject and identified by changes in density. Such cells can be identified by reference to previously known densities of the cancer cells or such densities can be identified using the methods disclosed herein. These references can be previously identified cancer cells obtained from American Type Culture Collection (Manassas, Va.) or cells obtained from other patients and tested using the methods and devices disclosed herein.

The methods disclosed herein can be performed using a device comprising a pair of permanent magnets. The magnets can be positioned, in a Helmholtz or anti-Helmholtz configuration, to provide a magnetic field of a predetermined field gradient. The device allows for a sample to be positioned between the pair of magnets. The sample holder is adapted for holding one or more samples in the magnetic field. In additional embodiments, the device includes a scale affixed to the magnet pair for use in determining the relative and/or absolute positions of organisms viewable in a sample. The scale can be a ruler.

In addition, if a component is not identified at a predetermined position, then this is indicative that the organism is not in the sample. If the component is identified, then this is indicative that the organism is in the sample.

The principle of magnetic levitation involves subjecting organisms having different densities in a fluid medium (or which develop different densities over time) having paramagnetic or superparamagnetic properties (a separating solution) to an inhomogeneous magnetic field. The magnetic field gradient interacts with the paramagnetic ions in the solution, as the paramagnetic ions are attracted to regions of higher magnetic field. The movement of paramagnetic ions toward the magnet displaces volume in the solution that the diamagnetic object, such as an organism, occupies. Accordingly, it appears that the diamagnetic object is repelled from the magnets or regions of high magnetic field. However, this is merely a by-product of the paramagnetic ions attraction to the magnetic fields.

In a non-limiting example of how magnetic levitation works, an object that is denser than the paramagnetic solution will sink, while an object that is less dense will rise in the solution. When the container comprising the solution with the objects is placed into a magnetic field, the paramagnetic ions move toward the magnets. This movement levitates the denser object to a position in the container that could be above its previous position. The movement of paramagnetic ions also levitates the less dense object to another position in the container, potentially to a lower position in the container. This phenomenon can be used to detect the particular density of an organism and other properties based on the organism's characteristic location in a magnetic fluid.

Organisms can exhibit very subtle differences in density and, thus, can occupy unique locations in a magnetic field gradient at equilibrium. This difference may be used to separate organisms of different densities, to identify the presence of a specific organism in a sample, to monitor the development or life cycle of an organism and to determine the physical state of the organism.

In one or more embodiments, differences in density of no more than 0.05 g/cm³, or even densities with accuracies of +/−0.0002 g/cm³ are detected or distinguished. Higher resolution is expected with optimization of the methods and devices according to one or more embodiments. In one or more embodiments, differences in density are used to detect and/or distinguish between organisms with and without labeling. Such labeling includes compounds that label fatty acids, lipids, carbohydrates, nucleic acids, and proteins. Exemplary labels include, but are not limited to, fluorescent labels, metallic particles, chemiluminescent labels, and radiolabels. The labels can be conjugated to different functional groups or to antibodies or fragments thereof (e.g., F_{ab} fragments). In addition, organisms can be complexed to compounds that do not label the organism, but change its density in a predetermined manner.

There are certain principles associated with density-based separations of diamagnetic materials. Density-based separations are determined by the balance between the magnetic force and the buoyant force on a diamagnetic organism in a paramagnetic solution. In a static system, the force per unit volume ( [symbol] ) on a organism in a magnetic field is the sum of the gravitational and magnetic forces (Equation 1),

$$\frac{{F}^{r}}{V} = ( {ρ}_{l} - {ρ}_{p} ) {g}^{r} - \frac{( {χ}_{l} - {χ}_{p} )}{{μ}_{o}} {( {B}^{r} \cdot {∇}^{r} ) {B}^{r}}^{•} \qquad ( 1 )$$

where the density of the liquid is ρ₁, the density of the organism is ρₚ, the acceleration due to gravity is g, the magnetic susceptibilities of the liquid and the organism are χ₁ and χₚ, respectively, the magnetic permeability of free space is μ₀, and the local magnetic field is B=(Bₓ, B_{y}, B_{z}). Both the magnetic field and its gradient contribute to the magnetic force and are optimized according to the dimensions of the system in order to maximize the separation. Equation 1 can be simplified for the levitation of a point organism—i.e., an infinitesimally small organism—in a system at equilibrium in which the magnetic field only has a vertical component (B_{z}); that is, the two other normal components of the applied magnetic field (B_{y} and B_{y}) are zero (Equation 2).

$$( {ρ}_{l} - {ρ}_{p} ) {g}^{r} = \frac{( {χ}_{l} - {χ}_{p} )}{{μ}_{o}} ( {B}_{z} \frac{\partial {B}_{z}}{\partial z} ) \qquad ( 2 )$$

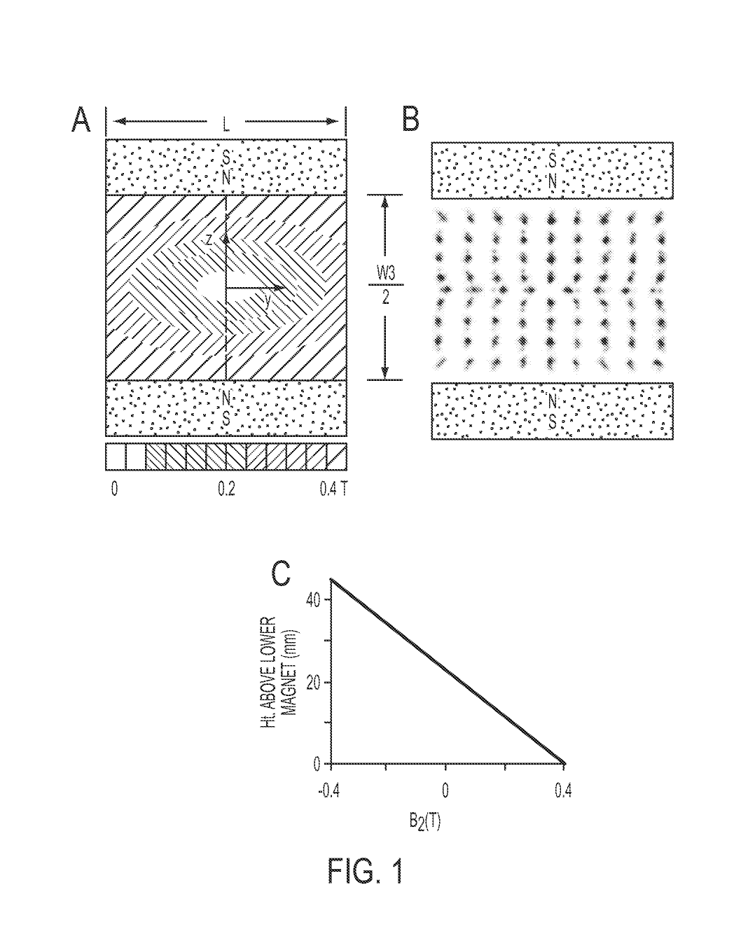

The distribution of magnetic field is determined by the size, geometry, orientation, and nature or type of the magnets. In specific embodiments, NdFeB magnets with length, width, and height of 5 cm, 5 cm, and 2.5 cm, respectively, having a magnetic field of about 0.4 T at their surface, are used to generate the required magnetic field and magnetic field gradient. In certain embodiments, the two magnets are oriented with like poles facing towards each other in the design of an anti-Helmholtz coil to establish the magnetic field distribution. In this geometry, the Bₓ and B_{y} components of the magnetic field are exactly zero only along the axis of the magnets, that is, along the vertical dashed line in FIG. 1A, as confirmed by the completely vertical orientation of the force along this axis. FIG. 1B illustrates the distribution of magnetic forces on a diamagnetic object within a paramagnetic solution. The calculation shows that a diamagnetic organism would be displaced from the surfaces of the magnets and would be trapped between the magnets, along the z-axis. The B_{z} component of the magnetic field also becomes zero over this axis, but only at the midpoint between the two magnets. The effect of the magnetic force in this geometry is to attract the paramagnetic solution towards one or the other of the two magnets and, as a consequence, to trap all diamagnetic organisms at the central region between the magnets (FIG. 1B)—i.e., where B_{z} is close to zero.

For this particular configuration, when the distance between the two magnets is

$$\frac{l \sqrt{2}}{3}$$

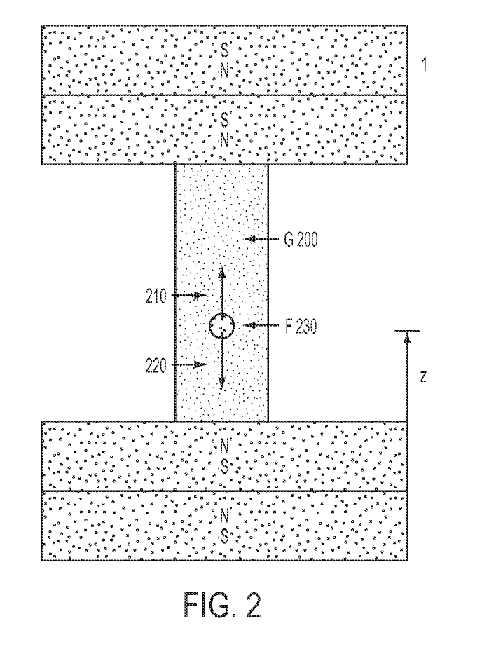

times the length (l) of the magnets, the magnetic field profile is approximately linear, and the gradient of the magnetic field is approximately constant in the z-direction (FIG. 1C). FIG. 1C is a graph of the calculated magnitude of the magnetic field in the vertical direction, B_{z}, along the axis between the two magnets (the dotted line in FIG. 1A); the direction of a positive z-vector was chosen to be toward the upper magnet. The other components of the magnetic field along the chosen path are zero. Note that the gradient of the magnetic field in the vertical direction is constant—i.e., a constant slope in the variation of the magnetic field along the axis. Thus, organisms of different densities will align themselves along the z-axis in predictable spacings. An exemplary system is illustrated in FIG. 2. A magnetic solution (200) is disposed between two magnets. Magnetic force and gravity are indicated by arrows (210 and 220) illustrating the opposing direction of these two forces. A diamagnetic organism (230) will reach an equilibrium position within the magnetic field. In one or more embodiments, this configuration is used for separating materials that differ in density.

In one or more embodiments, the solution has a positive magnetic susceptibility. The solvent used for the liquid solution should not damage or kill the organism to be separated from the other components in the solution. Typical liquids include water and other non-toxic polar solvents, such as salt solutions and Percoll dissolved in water. In certain embodiments, deuterium oxide (i.e., “heavy water”) or a mixture of deuterium oxide and water is used as the solvent. The density of the solution determines the objects that can and cannot be levitated. The magnetic susceptibility of the solution determines the separation resolution possible. That is, in an iso-dense solution, there is a large separation in solutions with a lower concentration of paramagnetic salts. The separation distance between two levitating objects in the magnetic field decreases as the concentration of paramagnetic salt increases. For example, by selecting a solvent that is more or less dense than the organism to be separated, the organisms will either sink or float prior to exposure to the magnetic field gradient. Solvent density may be selected such that all the organisms float or sink prior to the separation process. The solubility of the paramagnetic salt in the solvent is also a consideration.

### EXAMPLES

### Example 1

### Determination of Density of C. elegans

To show the applicability of the present methodologies, two experiments using magnetic levitation (MagLev) to quantify the change in density in different organisms are described. In particular, experiments were performed on C. elegans and embryos of Danio rerio (i.e., zebrafish). In these embodiments, the paramagnetic salt was chelated Mn•EDTA, and the osmolality of the paramagnetic medium was approximately isotonic with the species under study (˜300 mOsm/kg).

In the experiments on C. elegans, the organism was exposed to aspirin, which results in an accumulation of lipids in the organism due to the sequestration of coenzyme A and the inhibition of fatty acid degradation. A density estimate of C. elegans exposed to aspirin and the density of control C. elegans that were not exposed to aspirin was determined via a Percoll gradient. The density of C. elegans changed upon exposure to aspirin with respect to an unexposed control group. Aspirin-treated C. elegans were centrifuged with a set of density marker beads in order to measure the density of different populations of C. elegans qualitatively and to establish the ranges of interest for quantitative density measurements using MagLev. A chelated form of manganese, ethylenediaminetetraacetic acid disodium manganese salt (Mn•EDTA), was used in the MagLev experiments as it is FDA approved for in vivo applications. The analysis of treated and untreated populations of C. elegans by MagLev employed concentrations of Mn•EDTA up to several hundred millimolar. C. elegans were motile within the paramagnetic solution and their swimming motion counteracted the balance of magnetic and gravitational forces within the MagLev device. Ivermectin was introduced into the paramagnetic solution to paralyze the organisms, this stabilized the organisms within the MagLev device.

Although the density of C. elegans and other organisms can be assessed by centrifugation in Percoll gradients, these gradients can lead to physiological damage and death. Such gradients are ineffective for the analysis of living organisms. Thus, magnetic levitation offers an ideal solution for measuring changes in density easily in a manner that does not kill organisms and allows the examination of changes in density in long-term experiments.

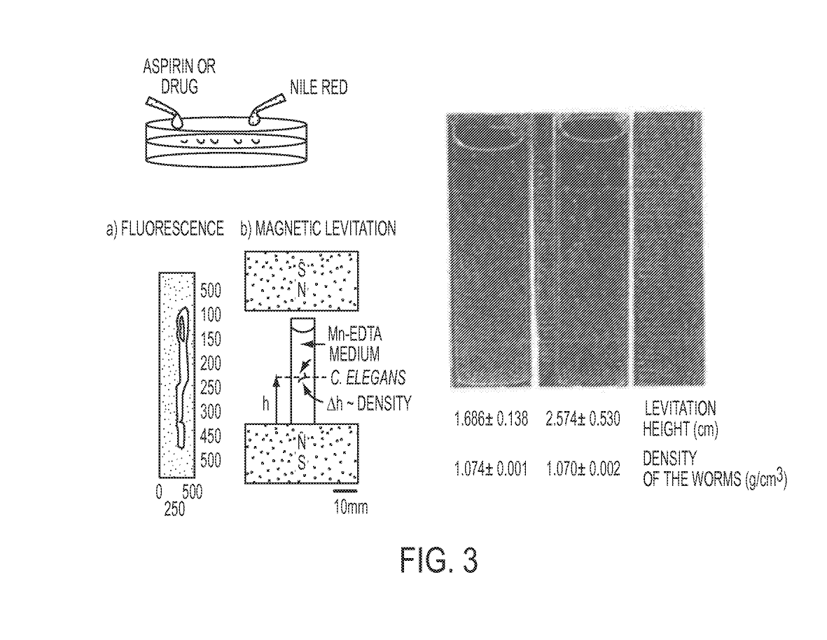

Using MagLev, the density of different populations of C. elegans was calculated with high precision. For example, worms treated with 6 mM aspirin levitated at a lower density, 1.070±0.002 g/cm³, than untreated C. elegans, 1.074±0.001 g/cm³ (see FIG. 3). FIG. 3 shows the effects on density due to the exposure of worms to different drugs. In these experiments, the lipophilic dye Nile Red (Nr) enabled the visualization of the stored fat within the bodies of the worms following exposure to different drugs. The magnetic levitation set up used to quantify the density of each worm involved placing the sample between two magnets. The density value is proportional to the distance h between the bottom magnet and the position of C. elegans. (FIG. 3c-d). The images show the levitation heights of different populations of C. elegans after exposure to (c) Nile Red or (d) 6 mM aspirin and Nile Red. The head of each worm was identified by a yellow dot using Photoshop.

In these experiments, the medium of levitation is 33% Percoll, 67% M9 buffer, 135 mM Mn•EDTA and 0.057 mM Ivermectin. The densities of the worms were calculated using B₀ (0.4 T), a distance between magnets of 4.5 cm, and T=23° C. Values are the average of the height or density calculated for each worm in each cuvette (N=10 worms).

### Example 2

### Monitoring Development of Zebrafish

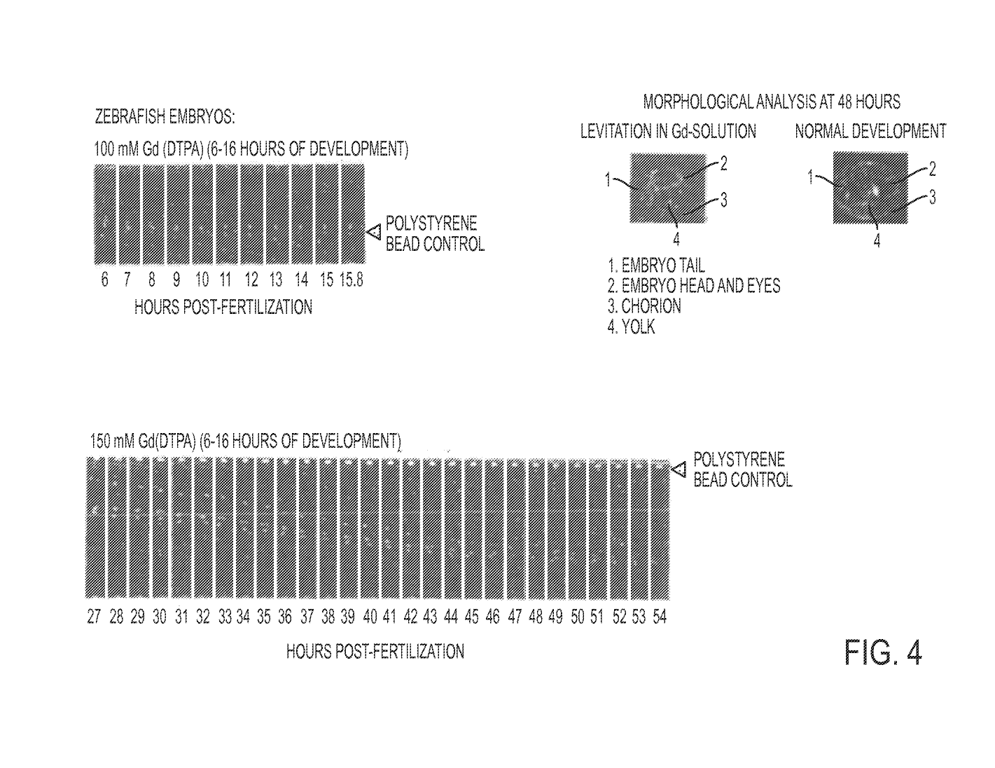

The development of zebrafish was monitored over a period of 54 h in a solution of 100 mM Gd•DTPA and 150 mM Gd•DTPA with Percoll and a saline solution (FIG. 4). The density of the embryos increased over time and their development was not affected by the paramagnetic solution used in the experiments. In these experiments, four zebrafish embryos (collected from one strain of fish) were placed into a paramagnetic medium containing Gd•DTPA, Percoll and saline buffer. For these experiments, polystyrene spheres were included as density controls. After 16 hours of monitoring development, the levitation medium was changed to one composed of a higher concentration of the gadolinium chelate. The increase in the concentration of the paramagnetic salt did not affect the morphology of the embryos. Pictured at the top right of FIG. 4 is a comparison between levitated zebrafish embryos and those that develop normally.

### Example 3

### Monitoring Development of C. Elegans in Microfluidic Devices

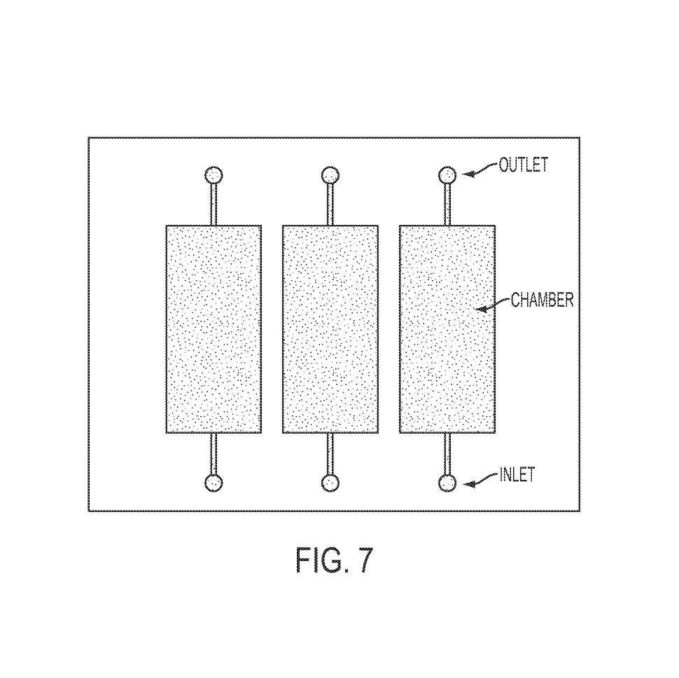

The microfluidic devices used for the magnetic levitation experiments of C. elegans is shown in FIGS. 5a-5b and 7. The devices comprise three chambers and each of them has an inlet and outlet channel to load and unload the paramagnetic solution with worms in and out of the chamber.

Regarding the actual loading and use of the microfluidic device, a first syringe with 10 mL of the paramagnet solution was prepared and was connected to a plastic tube. The tubing was inserted in the inlet of the chamber. The syringe was used to push the solution and fill up the chamber. Another plastic tube was connected to the outlet to conduct the excessive solution loaded to a waste container. After the solution was loaded, the syringe and plastic tubing was disconnected from the inlet of the chamber. A drop of 50 μL of M9 buffer which contained ˜10 worms was introduced in the inlet of the chambers. The syringe and plastic tubing with paramagnetic solution was reconnected and pressure was applied with the syringe to introduce the worms along with more paramagnetic solution into the chamber. This was done until all the worms were inside the chamber. The worms do not exit the chamber since the outlet channel was designed such that its width is smaller than the width of the worms. After the worms had been loaded, the inlet and outlet of the solutions were blanked with a plastic or glass rod.

We claim:

1. A method for detecting an effect of a compound of interest on a biological system, comprising:
contacting a test sample comprising an organism with the compound of interest;
applying a magnetic field to the test sample in a paramagnetic solution;
determining the density of the organism in the test sample, wherein the organism occupies a position in the magnetic field that corresponds to its density;
comparing the density or location of the organism in the test sample to a reference density or location of an untreated reference organism; and
detecting the effect of the compound of interest on a biological condition based on a change in density of the organism.

2. The method of claim 1, wherein location of the organism in the test sample is determined at different time points.

3. The method of claim 1, wherein the change in density in the organism is an indication of altered fat content when the organism is in the presence of the compound of interest.

4. The method of claim 1, wherein the change in density in the organism is indicative of uptake and accumulation of the compound of interest by the organism.

5. The method of claim 1, further comprising:
providing a plurality of test samples comprising the organism;
introducing a different compound of interest into each of the plurality of test samples; and
identifying those test samples containing organism contacted with the different compound of interest that demonstrate a change in density or location relative to the reference density or location of a reference organism that is not contacted with the different compound of interest, wherein the change in density or location is indicative of a biological effect on the organism.

6. The method of claim 1, wherein the organism is an embryo, a bacterium, a protist, an ova, a spermatozoa, a nematode, a eukaryotic cell, or combinations thereof.

7. The method of claim 1, wherein the organism is a plant tissue, a seed, a seedling, a tumor, a cancer mass, a group of cells, a spore, a pollen granule, a worm, or a multicellular parasite.

8. The method of claim 6, wherein detecting the effect of the compound of interest is a change in embryonic development.

9. The method of claim 5, further comprising separating the plurality of samples using a microfluidic device.

10. A method for determining the toxicity of a compound on a biological system, comprising:
contacting a plurality of test samples comprising an organism to a compound of interest at increasing concentrations;
applying a magnetic field to the test samples;
determining the density of the organism in the plurality of test samples, wherein the organism occupies a position in the magnetic field that corresponds to its density;
identifying the density in the test sample with a level of altered fat content of the organism, wherein a preselected level of fat content is associated with toxicity; and
determining a concentration of the compound of interest that provides a density in the organism associated with toxicity.

11. A method of evaluating an embryo, comprising:
exposing a paramagnetic solution comprising an embryo to a magnetic field, wherein the embryo occupies a position in the magnetic field that is an indication of its density;
monitoring the position of the embryo with time; and
detecting a change in location over time, the change in location being associated with gestational development of the embryo.

12. The method of claim 11, wherein the change in density or position identifies a change in gestational growth rate.

13. A method of sorting a population of organisms, comprising:
exposing a paramagnetic solution comprising a population of organisms to a magnetic field, wherein individual members of the population occupy positions in the magnetic field that correspond to their densities; and
sorting the population based on its position in the magnetic field.

14. The method of claim 13, further comprising isolating the population from the paramagnetic solution.

15. A method of analyzing a sample for the presence of an organism, comprising:
exposing a test sample to a magnetic field;
determining positions in the magnetic field of one or more constituent components of the test sample, wherein the positions are characteristic of their densities; and
detecting the presence or absence of a component at a predetermined position in the magnetic field that is associated with the presence or absence of the organism in the test sample.

16. The method of claim 15, wherein the sample is a biological sample.

17. The method of claim 16, wherein the biological sample is selected from the group consisting of bodily fluids and body tissues.

18. The method of claim 15, wherein the organism has been preselected based on a characteristic of the organism.

19. The method of claim 18, wherein the preselected organism is a parasite and the presence of the organism in the sample is indicative of parasitic infection.

20. A method of analyzing an organism of interest, comprising:
providing a paramagnetic solution of a composition and osmolality compatible with an organism of interest;
introducing the organism of interest into the paramagnetic solution;
applying a magnetic field to the paramagnetic solution; and
detecting density of the organism of interest by determining the position of the organism of interest in the magnetic field.

21. The method of claim 20, wherein the paramagnetic solution comprises a chelated paramagnetic salt.

22. The method of claim 21, wherein the chelated paramagnetic salt comprising manganese.

23. The method of claim 20, wherein the paramagnetic solution further comprises a paralyzing agent.

24. The method of claim 20, wherein the temperature of the paramagnetic solution is lower than the optimal temperature of the organism.

25. The method of claim 20, wherein the organism is selected from the group consisting of prokaryotic cells, eukaryotic cells, parasitic worms, ova, embryos and spermatozoa.

26. The method of claim 20, wherein the organism is a plant tissue, a seed, a seedling, a tumor, a cancer mass, a group of cells, a spore, a pollen granule, a worm, or a multicellular parasite.

27. A device for determining the effect of a compound on a biological system, comprising:
a pair of permanent magnets positioned to provide a magnetic field of a predetermined field gradient;
a sample holder located within the magnetic field for receiving a sample comprising an organism; and
a scale affixed to the magnet pair for use in determining the relative and/or absolute positions of organisms viewable in a sample.

28. The device of claim 27, wherein the sample is configured to receive a sample comprising a suspension of organisms housed in a microfluidic chip.

29. The method of claim 1, wherein the change in density in the organism is an indication of altered water content when the organism is in the presence of the compound of interest.
